(19) United States
(12) Patent Application Publication
(10) Pub. No.: US 2024/0335506 A1
(43) Pub. Date: Oct. 10, 2024

(54) AQP1 GENE THERAPY TO PREVENT RADIATION INDUCED SALIVARY HYPOFUNCTION

(71) Applicant: The United States of America,as represented by the Secretary,Department of Health and Human Services, Bethesda, MD (US)

(72) Inventors: John A. Chiorini, Dayton, MD (US); Matthew P. Hoffman, Bethesda, MD (US)

(73) Assignee: The United States of America,as represented by the Secretary,Department of Health and Human Services, Bethesda, MD (US)

(21) Appl. No.: 18/294,048

(22) PCT Filed: Aug. 4, 2022
(86) PCT No.: PCT/US2022/074559

§ 371 Date: Jan. 31, 2024

Related U.S. Application Data
(60) Provisional application No. 63/297,342, filed on Jan. 7, 2022.
(60) Provisional application No. 63/229,279, filed on Aug. 4, 2021.

(51) Int. Cl. A61K 38/17; A61K 48/00; A61N 5/10; A61P 1/00; C12N 15/86

(52) U.S. Cl. CPC A61K 38/1709; A61K 48/0033; A61K 48/0075; A61P 1/00; C12N 15/86; A61N 5/10; C12N 2750/14143

(57) ABSTRACT

Administration of aquaporin-1 (AQP1) complementary deoxyribonucleic acid (cDNA) to a salivary gland prior to treatment with ionizing radiation (IR) prevents the subsequent IR-induced loss in function. The administration of AQPI (e.g., human AQP1: hAQP1) prior to IR treatment (e.g., in patients with head and neck cancer) can reduce or prevent IR-induced salivary hypofunction, resulting in an elevated salivary output.

### CROSS-REFERENCE TO RELATED APPLICATIONS

This patent application claims the benefit of U.S. Provisional Patent Application No. 63/229,279, filed Aug. 4, 2021, which is incorporated by reference. This patent application also claims the benefit of U.S. Provisional Patent Application No. 63/297,342, filed Jan. 7, 2022, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 6,274 byte file named “763111.xml,” dated Aug. 4, 2022.

### BACKGROUND OF THE INVENTION

Common treatment for head and neck cancer patients involves ionizing radiation (IR). However, administration of IR to these patients damages salivary glands, which suffer irreversible damage, negatively impacting patients' quality of life.

No conventional therapy exists for this condition. However, it has been found that administration of a vector encoding aquaporin-1 (AQP1) after radiation therapy can affect the repair and reengineering of salivary glands. While effective in some respects, this process can result in considerable pain and suffering to the patient, as well as significant oral health problems. Accordingly, there remains a need for an improved method for treatment of patients undergoing IR treatment, such as head and neck cancer patients, to combat the negative impact of IR therapy on salivary glands.

### BRIEF SUMMARY OF THE INVENTION

In accordance with the present invention, it has been surprisingly discovered that, by administering a vector encoding AQPI before irradiation, the detrimental effects of IR therapy can be reduced or prevented. Administration of AQPI complementary deoxyribonucleic acid (cDNA) to a salivary gland prior to treatment with IR prevents the subsequent IR-induced loss in function. The administration of AQPI (e.g., human AQP1: hAQP1) prior to IR treatment (e.g., in patients with head and neck cancer) can reduce or prevent IR-induced salivary hypofunction, resulting in an elevated salivary output.

Accordingly, in accordance with an aspect, the invention provides a vector (e.g., AAV vector) and a virion comprising such a vector (e.g., AAV virion) that encodes an AQP1 protein for the prevention or reduction of radiation-induced salivary dysfunction (e.g., hypofunction) in a subject. The use of the vector or virion for the preparation of a medicament to prevent or reduce radiation-induced salivary dysfunction in a subject also is provided. In one aspect, such a vector or virion is useful for protecting a subject from radiation-induced salivary dysfunction.

In accordance with an aspect, the invention provides a method of preventing or reducing radiation-induced salivary dysfunction in a subject. The method comprises (a) administering a vector encoding an aquaporin (AQP) protein to the subject, and (b) administering ionizing radiation to the subject following (a), thereby preventing or reducing radiation-induced salivary dysfunction in the subject. In one aspect, salivary gland function can be maintained at a level equivalent to, or at least equivalent to, salivary gland function prior to administration of the ionizing radiation.

The AQP protein can be any suitable AQP protein including but not limited to an AQP1 protein. For example, in one aspect, the AQPI protein is or comprises human AQP1 (hAQP1) protein.

The vector encoding the AQP (e.g., hAPQP1) can be or comprise any suitable vector including but not limited to a viral vector. For example, in one aspect, the viral vector is or comprises an adenoviral vector (e.g., serotype 2 or serotype 5 adenoviral vector). In another aspect, the viral vector is or comprises an adeno-associated viral (AAV) vector (e.g., AAV2, AAV5, AAV6, AAV44.9, or BAAV).

The viral vector (e.g., AAV vector) can be administered to the subject as a vector or as a virion comprising the vector (e.g., AAV vector). In one aspect, the virion is or comprises an AAV virion. The vector or virion can be administered to the subject at any suitable location and by any suitable administration route. In aspect, the vector or virion is administered to a salivary gland of the subject.

### BRIEF DESCRIPTION OF THE SEVERAL VIEWS OF THE DRAWING(S)

FIG. 1 is a graph demonstrating saliva flow in AQPI treated mice. Saliva flow is presented as microliters/gm body weight.

FIG. 2 is a graph demonstrating saliva flow in AQPI treated mice. Saliva flow is presented as microliters/gm body weight.

FIG. 3 is a graph demonstrating saliva flow in AQPI treated mice Saliva flow is presented as microliters/gm body weight.

FIG. 4 is a heatmap of salivary gland cells as analyzed by single cell RNAseq. Data from single cell RNAseq of either nonIR, GFP treated IR mice, or AQPI treated before (AQPI B) or after (AQPIA) were used to produce UMAPs and identify 16 distinct cell clusters (y-axis). Comparison of the distribution of cells between the different clusters in each of the 4 conditions was used to generate the heatmap.

FIGS. 5A-D are images from histology of mouse submandibular glands. The images were taken from whole scanned slides of generally the same area of each gland near the hilum. Dashed boxes on 5X delimit inset pictures to emphasize features including radiation-induced changes, fibrosis, atrophy, and inflammation. FIG. 5A corresponds to AAV-GFP before irradiation. FIG. 5B corresponds to AAV-AQPI before irradiation.

FIG. 5C corresponds to AAV-GFP after irradiation. FIG. 5D corresponds to AAV-AQP1 after irradiation.

### DETAILED DESCRIPTION OF THE INVENTION

The invention provides a method involving pretreating a subject (e.g., a human patient) undergoing IR treatment impacting the salivary (e.g., parotid, submandibular, or sublingual) glands with a vector encoding an aquaporin (AQP) protein. For example, the method can be applied to a subject undergoing IR treatment for cancers, such as head and neck cancer. Thus, in according to a first aspect, the invention provides a method of preventing or reducing radiation-induced salivary dysfunction in a subject, comprising: (a) administering a vector encoding an aquaporin (AQP) protein to the subject, and (b) administering ionizing radiation to the subject following (a), thereby preventing or reducing radiation-induced salivary dysfunction in the subject.

As used herein, an aquaporin protein, also referred to as AQP protein, can be or comprise any protein that exhibits activity of an exemplary aquaporin protein (e.g., human aquaporin (“hAQP”)), such the ability to form a channel that allows the passage of water. AQP proteins, nucleic acid, and associated vectors are known to those of skill in the art and are described, by way of non-limiting example, in U.S. Pat. No. 10,166,299, the entirety of which is incorporated by reference herein.

An AQP protein, in the context of the present invention, can have or comprise a wild-type (wt) AQP sequence (i.e., it has the same amino acid sequence as a natural AQP protein), be or comprise any portion of a wt AQP protein, or be or comprise a variant of the natural AQP protein, provided that such a portion or variant retains the ability to form a channel that allows the passage of water. Assays to determine the ability of an AQP protein of the present invention to form a channel that allows the passage of water are known to those skilled in the art (see, for example, Lui et al., Journal of Biological Chemistry, 281, 15485-15495 (2006), the entirety of which is incorporated by reference herein).

In one aspect, a protein useful in the methods of the present invention is an AQP1 protein comprising the entire amino acid sequence of a naturally occurring AQPI protein. Examples of human AQPI proteins include but are not limited to NCBI Reference No. NP_932766.1 (SEQ ID NO: 1), NCBI Reference No. NP_001171989.1 (SEQ ID NO: 2), and NCBI Reference No. NP_001171990.1 (SEQ ID NO: 3), and NP_001171991.1 (SEQ ID NO: 4). An example of a murine AQP1 protein includes but is not limited to SEQ ID NO: 5.

Examples of the AQP protein, nucleic acid, and associated vectors for use in the invention are described herein and in U.S. Pat. No. 10,166,299, the entirety of which is incorporated by reference.

In one aspect, an AQPI protein comprises a portion of the amino acid sequence of an AQPI protein, wherein such portion of an AQPI protein retains the ability to form a channel in a cell membrane that allows the passage of water. Several isoforms of AQPI protein exist. Thus, in one aspect, an AQP1 protein is or comprises an isoform of an AQP-protein, wherein such isoform retains the ability to form a channel that allows the passage of water. In one aspect, an AQPI protein is or comprises a portion of an isoform or other naturally-occurring variant of an AQPI protein, wherein such portion retains the ability to form a channel in a membrane that allows the passage of water. Methods to produce functional portions and variants of AQPI proteins, such as conservative variants, of AQP1 protein are known to those skilled in the art.

Also encompassed in the present invention are AQPI protein variants that have been altered by genetic manipulation. With regard to such variants, any type of alteration in the amino acid sequence is permissible so long as the variant retains at least one AQPI protein activity described herein. Examples of such variations include, but are not limited to, amino acid deletions, amino acid insertions, amino acid substitutions and combinations thereof. For example, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein.

As noted, isolated variant proteins of the present invention can also contain amino acid substitutions as compared to the wild-type AQPI protein disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

Desired amino acid substitutions (whether conservative or non-conservative) can be determined by those skilled in the art at the time such substitutions are desired. For example, amino acid substitutions can be used to identify important residues of the AQPI protein, or to increase or decrease the affinity of the AQPI proteins described herein. Thus, in one aspect, the AQPI protein variant comprises at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or any ranges of values thereof) amino acid substitution (e.g., conservative substitution) relative to the AQPI proteins described herein (e.g., SEQ ID NOs: 1-5). In one aspect, the AQPI protein comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the AQPI proteins described herein (e.g., SEQ ID NOs: 1-5).

While proteins of the present invention can consist entirely of the sequences disclosed herein, and the disclosed variants thereof, such proteins may additionally contain amino acid sequences that do not confer AQPI activity, but which have other useful functions. Any useful, additional amino acid sequence can be added to the isolated protein sequence, so long as the additional sequences do not have an unwanted effect on the protein's ability to form a channel that allows the passage of water. For example, isolated proteins of the present invention can contain amino acid sequences that are useful for visualizing or purifying the peptide. Such sequences act as labels (e.g., enzymes) or tags (e.g., antibody binding sites). Examples of such labels and tags include, but are not limited to, beta-galactosidase, luciferase, glutathione-s-transferase, thioredoxin, HIS-tags, biotin tags, and fluorescent tags. Other useful sequences for labeling and tagging proteins are known to those of skill in the art.

In addition to the modifications described above, isolated proteins of the present invention can be further modified, so long as such modification does not significantly affect the ability of the protein to form a channel that allows the passage of water. Such modifications can be made, for example, to increase the stability, solubility or absorbability of the protein. Examples of such modifications include, but are not limited to PEGylation, glycosylation, phosphory lation, acetylation, myristylation, palmitoylation, amidation and/or other chemical modification of the peptide.

An AQPI protein can be derived from any species that expresses a functional AQPI protein. An AQPI protein can comprise the sequence of a human or other mammalian AQPI protein or a portion thereof. Additional examples include, but are not limited to, murine, feline, canine, equine, bovine, ovine, porcine or other companion animal, other zoo animal, or other livestock AQPI proteins. In one aspect, an AQPI protein comprises the amino acid sequence of a human AQPI protein or portion thereof. In another aspect, an AQPI protein comprises the amino acid sequence of a murine AQPI protein or a portion thereof.

In one aspect, an AQPI protein is joined to a fusion segment: such a protein is referred to as an AQPI fusion protein. Such a protein comprises an AQPI protein domain (also referred to herein as AQPI domain) and a fusion segment. A fusion segment is an amino acid segment of any size that can enhance the properties of AQPI protein. For example, a fusion segment of the invention can increase the stability of an AQPI fusion protein, add flexibility, or enhance or stabilize multimerization of the AQPI fusion protein. Examples of fusion segments include, without being limited to, an immunoglobulin fusion segment, an albumin fusion segment, and any other fusion segment that increases the biological half-life of the protein, provides flexibility to the protein, and/or enables or stabilizes multimerization. It is within the scope of the disclosure to use one or more fusion segments. Fusion segments can be joined to the amino terminus and/or carboxyl terminus of AQPI protein of the invention. As used herein, “join” refers to combine by attachment using genetic engineering techniques. In such an aspect, a nucleic acid molecule encoding an AQPI protein is physically linked to a nucleic acid molecule encoding a fusion segment such that the two encoding sequences are in frame and the transcription product forms a continuous fusion protein. In one aspect, an AQPI protein can be joined directly to a fusion segment, or an AQPI protein can be linked to the fusion segment by a linker of one or more amino acids.

Nucleic acid molecules (polynucleotides) that encode the AQPI proteins (e.g., AQPI fusion proteins) described herein also are provided as an aspect of the present invention. The nucleic acid molecules can comprise DNA, cDNA, and/or RNA, can be single or double stranded, and can be naturally-occurring, synthetic, and/or recombinant.

The polynucleotide can comprise nucleotide analogues or derivatives (e.g., inosine or phophorothioate nucleotides and the like). Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG: serine can be encoded by TCT, TCC, TCA, TCG, AGT, or AGC: asparagine can be encoded by AAT or AAC: aspartic acid can be encoded by GAT or GAC: cysteine can be encoded by TGT or TGC: alanine can be encoded by GCT, GCC, GCA, or GCG: glutamine can be encoded by CAA or CAG: tyrosine can be encoded by TAT or TAC; and isoleucine can be encoded by ATT, ATC, or ATA.

The polynucleotide can be provided, as part of a construct comprising the polynucleotide and elements that enable delivery of the polynucleotide to a cell, and/or expression of the polynucleotide in a cell. For example, the polynucleotide sequence encoding AQPI can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA and stop codons. Suitable promoters include, but are not limited to, an SV40 early promoter, RSV promoter, adenovirus major late promoter, human CMV immediate early I promoter, poxvirus promoter, 30K promoter, 13 promoter, sE/L promoter, 7.5K promoter, 40K promoter, and CI promoter.

A polynucleotide encoding the AQPI or fusion protein can be cloned or amplified by in vitro methods, such as the polymerase chain reaction (PCR), the ligase chain reaction (LCR), the transcription-based amplification system (TAS), the self-sustained sequence replication system (3SR) and the Q [symbol] ≤ replicase amplification system (QB). For example, a polynucleotide encoding the zinc finger protein can be isolated by polymerase chain reaction of cDNA using primers based on the DNA sequence of the molecule. A wide variety of cloning and in vitro amplification methodologies are well known to persons skilled in the art.

The vector for use in the invention includes plasmids (e.g., DNA plasmids), bacterial vectors, and viral vectors, such as adenovirus vectors, adeno-associated virus (AAV) vectors, poxvirus vectors, retrovirus vectors, herpes virus vectors, polio virus vectors, and alphavirus vectors. When the vector is a plasmid (e.g., DNA plasmid), the plasmid can be complexed with chitosan.

In one aspect, the vector is or comprises a viral vector, such as an adenoviral vector (e.g., serotype 2 or serotype 5) or an adeno-associated viral (AAV) vector. Such an AAV vector can be selected from an AAV1 vector, an AAV2 vector, an AAV3 vector, an AAV4 vector, an AAV5 vector, an AAV6 vector, an AAV7 vector, an AAV8 vector, an AAV9 vector, an AAV10 vector, an AAV11 vector, an AAV12 vector, an AAV44.9 (as described in U.S. Patent Application Publication No. 2018/0355376, which is incorporated herein in its entirety, and in the aforementioned U.S. Pat. No. 10,166,299), an AAAV vector, and a BAAV vector, wherein any of such vectors encode an AQP1 protein as described herein.

In one aspect, the AAV vector is or comprises an AAV2 vector, an AAV5 vector, an AAV6 vector, or a BAAV vector, wherein the respective vector encodes an AQPI protein as described herein. In one aspect, the AAV vector comprises AAV ITRs and a CMV promoter operatively linked to a nucleic acid molecule encoding an AQPI protein.

Plasmid vectors that encode an AQPI protein also are provided. Such plasmid vectors also can include control regions, such as AAV ITRs, a promoter operatively linked to the nucleic acid molecule encoding the AQPI protein, one or more splice sites, a polyadenylation site, and a transcription termination site. Such plasmid vectors also typically include a number of restriction enzyme sites as well as a nucleic acid molecule that encodes drug resistance.

The present invention also provides an AAV virion. As used herein, an AAV virion comprises an AAV vector encoding an AQP1 protein of the invention encapsidated in an AAV capsid. Examples of AAV capsids include AAV1 capsids, AAV2 capsids, AAV3 capsids, AAV4 capsids, AAV5 capsids, AAV6 capsids, AAV7 capsids, AAV8 capsids, AAV9) capsids, AAV10 capsids, AAV11 capsids, AAV12 capsids, AAV44.9 capsid, AAAV capsids, BAAV capsids, and capsids from other AAV serotypes known to those skilled in the art. In one aspect, the capsid is a chimeric capsid, i.e., a capsid comprising VP proteins from more than one serotype. As used herein, the serotype of an AAV virion of the invention is the serotype conferred by the VP capsid proteins. For example, an AAV2 virion is a virion comprising AAV2 VP1, VP2 and VP3 proteins. Any AAV virion can be used to practice the methods of the invention so long as the virion is capable of efficiently transducing ductal or acinar cells of salivary glands.

In one aspect, the AAV virion is selected from an AAV2 virion, an AAV5 virion, an AAV6 virion, and a BAAV virion, wherein the AAV vector within the virion encodes an AQPI protein.

Methods useful for producing AAV vectors and AAV virions disclosed herein are known to those skilled in the art. Briefly, an AAV vector of the present invention can be produced using recombinant DNA or RNA techniques to isolate nucleic acid sequences of interest and join them together as described herein, e.g., by using techniques known to those skilled in the art, such as restriction enzyme digestion, ligation, PCR amplification, and the like. Methods to produce an AAV virion of the invention typically include (a) introducing an AAV vector of the invention into a host, (b) introducing a helper vector into the host cell, wherein the helper vector comprises the viral functions missing from the AAV vector and (c) introducing a helper virus into the host cell. All functions for AAV virion replication and packaging need to be present, to achieve replication and packaging of the AAV vector into AAV virions. In some instances, at least one of the viral functions encoded by the helper vector can be expressed by the host cell. Introduction of the vectors and helper virus can be carried out using standard techniques and occur simultaneously or sequentially. The host cells are then cultured to produce AAV virions, which are then purified using standard techniques, such as CsCl gradients. Residual helper virus activity can be inactivated using known methods, such as heat inactivation. Such methods typically result in high titers of highly purified AAV virions that are ready for use.

An AAV vector of a specified serotype can be packaged in a capsid of the same serotype. For example, an AAV2 vector can be packaged in an AAV2 capsid. In other instances, an AAV vector of a specified serotype is packaged in a capsid of a different serotype in order to modify the tropism of the resultant virion. Combinations of AAV vector serotypes and AAV capsid serotypes can be determined by those skilled in the art.

The vector for use in the invention can include an expression control sequence operatively linked to coding sequence, such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The term “enhancer” as used herein, refers to a DNA sequence that increases transcription of, for example, a nucleotide sequence to which it is operably linked. Enhancers can be located many kilobases away from the coding region of the nucleotide sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. For example, the nucleotide encoding the polypeptide can be operably linked to a CMV enhancer/chicken β-actin promoter (also referred to as a “CAG promoter”). Additionally, the vector can comprise nucleic acid sequence encoding a reporter to identify the transfection/transduction efficiency of the vector.

Compositions comprising the vector (e.g., AAV vector) encoding an AQP protein also are provided. Compositions comprising an AAV virion comprising an AAV vector encoding an AQPI protein also are provided. Such compositions can comprise a carrier (e.g., a pharmaceutically or physiologically acceptable carrier). For example, such compositions can include an aqueous solution, such as a physiologically compatible buffer. Examples of excipients to be included in the compositions include water, saline, Ringer's solution, and other aqueous physiologically balanced salt solutions. In some aspects, excipients are added to, for example, maintain particle stability or to prevent aggregation. Examples of such excipients include, but are not limited to, magnesium to maintain particle stability, pluronic acid to reduce sticking, mannitol to reduce aggregation, and the like, known to those skilled in the art.

Compositions are conveniently formulated in a form suitable for administration to a subject. Techniques to formulate such compositions are known to those skilled in the art. For example, the vector (e.g., an AAV vector) or virion of the invention can be combined with saline or other pharmaceutically acceptable solution. In some aspects, excipients are also added. In another aspect, a composition comprising a vector (e.g., AAV vector) or virion is dried, and a saline solution or other pharmaceutically acceptable solution can be added to the composition prior to administration.

Furthermore, the vector or virion can be used in the methods described herein alone or as part of a pharmaceutical formulation.

The composition (e.g., pharmaceutical composition) can comprise one or more other additional therapeutic agents. Examples of such additional therapeutic agents that may be suitable for use in the composition include gene therapies, anti-inflammatoires, free radical scavengers, radioprotectants, and agents or drugs that increase saliva production.

The viral vector (e.g., AAV vector) can be administered to the subject as a vector or as a virion comprising the vector (e.g., AAV vector). In one aspect, the virion is an AAV virion. The vector or virion can be administered to the subject at any suitable location and by any suitable administration route. In aspect, the vector or virion is administered to a salivary gland of the subject.

As used herein, the ability of the vector or virion to prevent or reduce radiation-induced salivary dysfunction refers to the ability of such vector or virion to completely or partially eliminate radiation-induced salivary dysfunction. For example, with regard to the flow of saliva, methods of the present invention can return such flow to 70%, 80%, 85%, 90%, 95% or 100% of the value observed in a normal individual (i.e., an individual who has not been administered radiation).

The disclosure provides a method comprising administering the vector or virion to a subject, wherein such administration maintains salivary gland function in such a subject following radiation. As used herein, maintaining salivary gland function in a subject that has been administered the vector or virion means that salivary gland function after administration of radiation is equivalent (or at least equivalent) to salivary gland function in that subject prior to administration of the radiation. For example, after radiation of the subject to which the vector or virion has been administered, the salivary gland function in the subject does not worsen but is equivalent (or at least equivalent) to function prior to radiation. In an aspect of the invention, the method comprises (a) administering a vector encoding an AQP protein to a subject that has not been treated with ionizing radiation, and (b) administering ionizing radiation to the subject following (a), thereby preventing or reducing radiation-induced salivary dysfunction in the subject.

As used herein, a “subject” includes humans and other mammals, such as mice, rats, hamsters, cats, dogs, pigs, cows, horses, other companion animals, other zoo animals, lab animals (e.g., mice), and livestock.

The vector or virion can be administered in a variety of routes. In some aspects, the vector or virion is administered by aerosol. In some aspects, the vector or virion is administered to the mucosa. In some aspects, the vector or virion is administered directly to a tissue or organ. In some aspects, the vector or virion is administered to a salivary gland (e.g., a parotid, submandibular, or sublingual gland).

The invention also provides ex vivo methods to prevent or reduce radiation-induced salivary dysfunction. Such methods can involve administering the vector or virion to a cell, tissue, or organ outside the body of the subject, and then placing that cell, tissue, or organ into the body. Such methods are known to those skilled in the art.

In aspects, the invention provides a cell (e.g., a salivary gland cell), tissue, or organ transfected with an AAV vector that encodes an AQPI protein. The cell (e.g., salivary gland cell), tissue, or organ (e.g., a salivary gland, such as a parotid, submandibular, or sublingual gland) can be that of a subject that is planning to or has received radiation or a cell, tissue, or organ ex vivo.

The vector, virion, or composition (e.g., pharmaceutical composition) thereof can be administered alone or in combination with one or more other additional therapeutic agents. Examples of such additional therapeutic agents that may be suitable include gene therapies, anti-inflammatoires, free radical scavengers, radioprotectants, and agents or drugs that increase saliva production.

The dose of compositions disclosed herein to be administered to a subject to be effective (i.e., to prevent or reduce radiation-induced salivary dysfunction) will depend on the subject's condition, manner of administration, and judgment of the prescribing physician. An exemplary dose can range from about 10⁴ virion particles per kilogram to about 10¹² virion particles per kilogram of the subject (e.g., 10⁴, 10⁵, 10⁶, 10⁷, 10⁸, 10⁹, 10¹⁰, 10¹¹, 10¹², and ranges thereof). A preferred dose is in the range of from about 10⁶ virion particles per kilogram to about 10¹² virion particles per kilogram. A more preferred does is in the range of from about 10⁸ virion particles per kilogram to about 10¹² virion particles per kilogram.

An alternative exemplary dose can range from about 10⁴ virion particles per gram of the gland to about 10¹² virion particles per gram of the gland (e.g., about 10⁴, 10⁵, 10⁶, 10⁷, 10⁸, 10⁹, 10¹¹, 10¹² or ranges thereof).

In some aspects, the dose is determined by the amount of fluid needed to fill the gland. For vector to cell contact to occur, the gland should be filled with fluid in order to introduce the vector to the cell. In IR patients, volumes range from about 500 μL to about 2.5 mL (e.g., 500 μL, 600 μL, 700 μL, 800 μL, 900 μL, 1 mL, 1.1 mL, 1.2 mL, 1.3 mL, 1.4 mL, 1.5 mL, 1.6 mL, 1.7 mL, 1.8 mL, 1.9 mL, 2 mL, 2.1 mL, 2.2 mL, 2.3 mL, 2.4 mL, 2.5 mL, or ranges thereof), depending on atrophy and fibrosis.

The following examples further illustrate the invention but, of course, should not be construed as in any way limiting its scope.

### Example 1

This example demonstrates administration of a vector (an AAV vector) encoding AQP1 before and after IR treatment promotes salivary gland function.

All murine experiments were approved by the National Institute of Dental and Craniofacial Research (NIDCR) Animal Care and Use Committee. Eight-week-old female C3H mice (National Cancer Institute Animal Production Area) were used. Groups of 7-8 C3H mice were treated with AAV2 vectors encoding either GFP, AQP1, neurturin, or a combination of AAV2-AQP1+AAV2-neurturin 10 days before or 2 months post irradiation (IR).

In particular, the treatment groups included injection of AAV2-GFP (10¹⁰ vp/g) or AAV2-hAQP1 (10¹⁰ or 10⁷ vp/g) or AAV2NRTN (10⁶, 10⁸, or 10¹⁰ vp/g) 10 days pre-IR or 60 days post-IR. Baseline saliva (non-IR) was collected prior to vector delivery or IR. To irradiate the salivary glands, each animal was placed in a specially built Lucite jig. This jig immobilizes animals without the use of anesthetics and allows IR to the head and neck region only.

Mice were irradiated in 5 fractions (6 Gy/day for 5 days) using a Therapax DXT300 X-ray irradiator (Pantak). After IR, animals were removed from the jig, housed (5 animals per cage) in a climate- and light-controlled environment, and allowed free access to food and water. To deliver viral vectors into submandibular glands, mice were anesthetized with ketamine (60 mg/kg) and xylazine (8 mg/kg) intramuscularly, upon which vectors were delivered into both submandibular glands by retro-ductal infusion. During the cannulation, 0.5 mg/kg atropine was intramuscularly applied to inhibit saliva secretion in order to increase transduction efficiency.

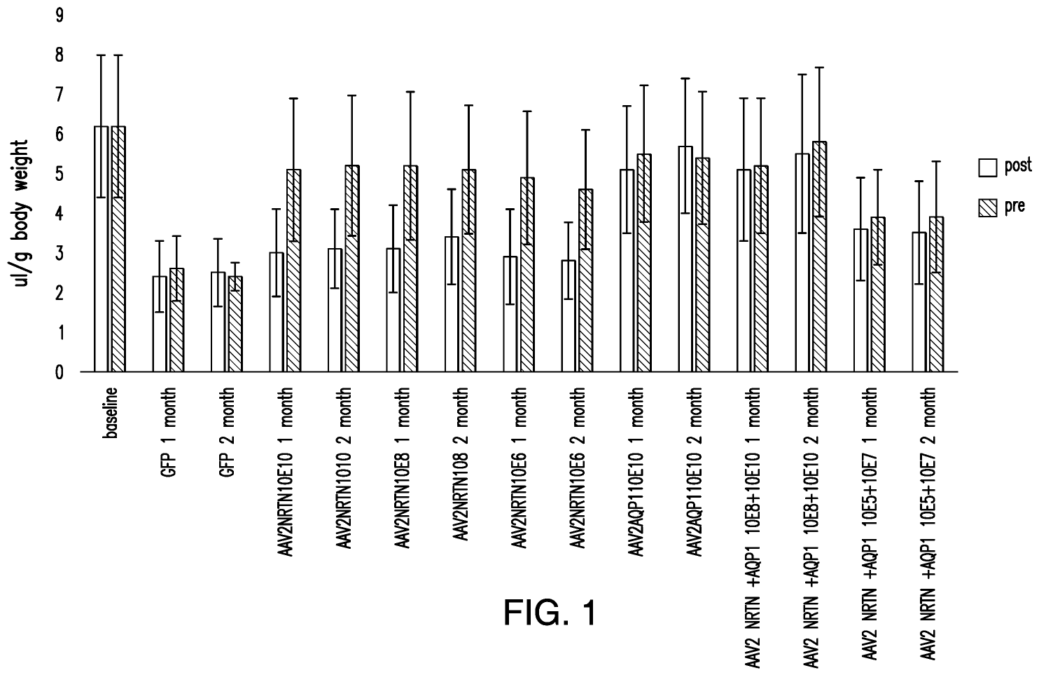

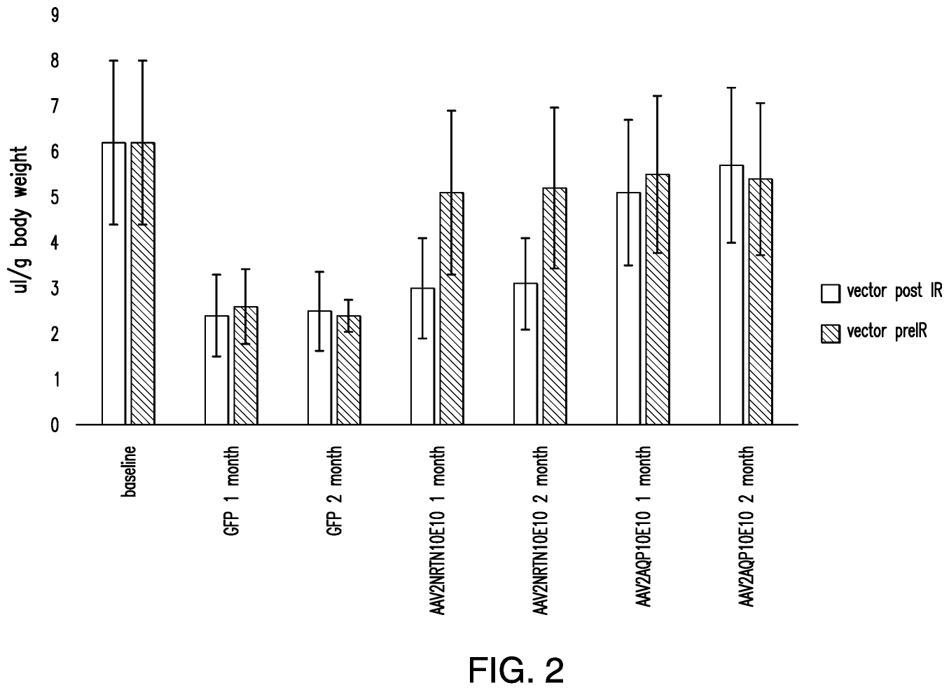

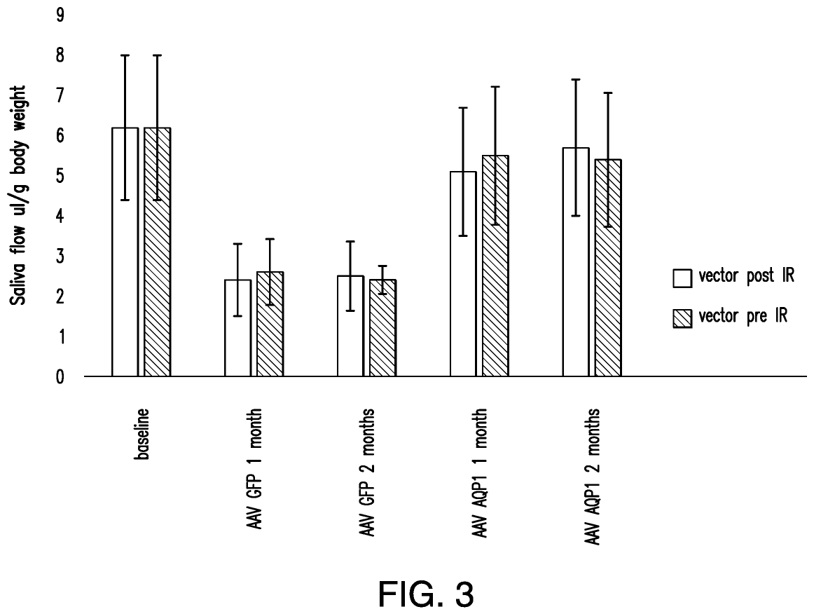

For saliva collections, mice were anesthetized as mentioned above, followed by subcutaneous injection of pilocarpine at 0.25 mg/kg body weight to stimulate saliva secretion. Whole saliva was collected with a 75-mm hematocrit tube (Drummond) into 1.5 mL pre-weight Eppendorf tubes for 20 min and frozen immediately. After 10 months, mice were sacrificed in a carbon dioxide chamber and the glands were removed for analysis. Saliva was collected from the mice prior to the start of experiment (baseline/non-IR). The results are presented in FIGS. 1-3.

The results show that compared to AAV2-GFP treated mice, AAV2-AQP1 treatment could prevent the loss of saliva flow if administered before IR or initiate the recovery of saliva flow following IR (p<. 01) (see FIGS. 1-3). In contrast, neurturin could only prevent the loss in saliva flow and could not initiate a substantial recovery. Furthermore, a combination of the two vectors was not synergistic and did substantially increase saliva flow beyond levels achieved with the AAV2AQPI vector alone administered pre- or post-IR.

These results support that administration of AAV-AQPI before and after IR treatment promotes salivary gland function.

Prior to the invention, the conventional understanding was that AQPI gene therapy required stable epithelial cells in order for AQPI expression to create a facilitated pathway for fluid movement. In clinical trials of AQP1, patients were required to be at least 2 years post IR treatment. As there is significant cell turnover and remodeling following IR treatment, it would be expected that AQPI AAV DNA would be lost from the transduced cells following IR treatment (i.e., not persist) and, therefore, could not create a facilitated pathway for fluid movement. For example, Malik et al., J. Virol., 71 (3): 1776-1783 (1997)), teaches that AAV can only persist in nondividing cells and is lost in a dividing population over time. Li et al., Int. J. Radiation Oncology Biol. Phys., 62 (5): 1510-1516 (2005)), teaches that there is significant remodeling in the gland that occurs following IR and the changes are followed by a loss in function. Additionally, Vitolo et al., Oral Diseases, 8:183-191 (2002)), teaches that AQPI gene therapy is for repair of the gland whereas other approaches are for prevention of IR damage to the gland. Vitolo et al. also discloses that salivary glands are a slowly dividing cell population and that AAV transduction can persist in the salivary gland.

Therefore, prior to the invention, AQPI gene therapy was not thought of as a prevention approach to IR-induced hypofunction because AQP1 gene therapy would not persist in a changing and remodeling environment like a salivary gland post-IR. However, as described herein, the invention disclosed herein—that the administration of AAV-AQP1 before and after IR treatment promotes salivary gland function—is surprising and unexpected.

### Example 2

This example characterizes the mechanism of saliva flow pre- and post-IR.

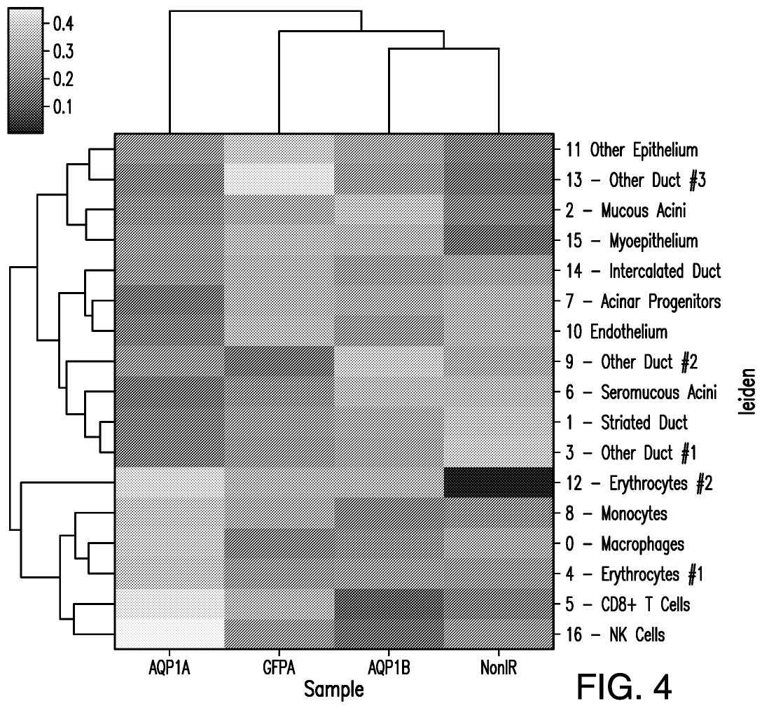

Data from single cell RNAseq of either nonIR, GFP treated IR mice, or AQP1 treated before (AQPIB) or after (AQPIA) was used to produce UMAPs and identify 16 distinct cell clusters. Comparison of the distribution of cells between the different clusters in each of the 4 conditions was used to generate the heatmap of FIG. 4.

AQPIB can be found in the same clade as the nonIR, while the GFP and AQPIA are in separate clades from each and from the AQP1B/nonIR clade. This result suggests that the cell populations are different between AQPIA and AQPIB. Furthermore, the clade organization of the cell type-based distribution in AQPIB is most similar to nonIR while AQPIA forms a distinct clade from this group and the IR effect GFP group.

These results support that saliva flow is the result of protection of the gland if given before IR: however, recovery of saliva flow is possible in a distinct cell population and environment if given after IR.

### Example 3

This example demonstrates that administration of AAV-AQPI before irradiation resulted in less pathological changes compared to the administration of AAV-AQPI after irradiation.

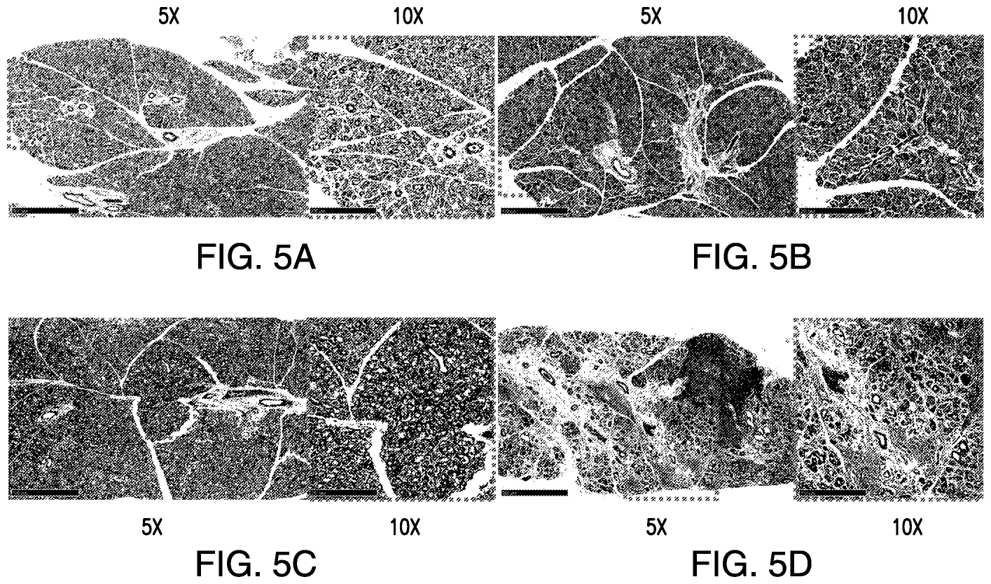

The histology of mouse submandibular gland was assessed. Images from mice administered AAV-GFP before irradiation (FIG. 5A), AAV-AQP1 before irradiation (FIG. 5B), AAV-GFP after irradiation (FIG. 5C), and AAV-AQP1 after irradiation (FIG. 5D) were taken from whole scanned slides of generally the same area of each gland near the hilum. Overall morphologic changes in the gland were assessed by H&E staining.

Using a grading scale of 0-3, sections were assessed for atropy, fibrosis, and immune infiltration. Collectively all had increased atrophy and fibrosis. They also had increased inflammation with intraglandular germinal center formation and reactive lymph nodes present within the gland capsule. There were increased numbers of multinucleate acinar cells and some areas of ductal hyperplasia.

However, the average score for the after-treatment group was 2.2+/−0.75 and the before treatment group was 1.5+/−. 54 (p>0.05). Therefore, the after IR treatment group tended to show more noteworthy pathological changes compared with the before IR treatment group (FIGS. 5A-D).

These results support that administering a vector encoding AQPI before irradiation reduces the detrimental effects observed when a vector encoding AQPI is administered after irradiation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Biological Sequences

The following sequences are referenced herein:

```
SEQ ID NO: 1:
    1 masefkkklf wravvaefla ttlfvfisig salgfkypvg nnqtavqdnv kvslafglsi
  61 atlaqsvghi sgahlnpavt lglllscqis ifralmyiia qcvgaivata ilsgitsslt
121 gnslgrndla dgvnsgqglg ieiigtlqlv lcvlattdrr rrdlggsapl aiglsvalgh
181 llaidytgcg inparsfgsa vithnfsnhw ifwvgpfigg alavliydfi laprssdltd
241 rvkvwtsgqv eeydldaddi nsrvemkpk
SEQ ID NO: 2:
    1 mpgarplplv lvpqntlawm qldakapahp rplqllgrvg pgsrqladgv nsgqglgiei
  61 igtlqlvlcv lattdrrrrd lggsaplaig lsvalghlla idytgcginp arsfgsavit
121 hnfsnhwifw vgpfiggala vliydfilap rssdltdrvk vwtsgqveey dldaddinsr
181 vemkpk
SEQ ID NO: 3:
    1 mfwtfgyeav spagpshlfa slllgvllti tfmpgarplp lvlvpqntla wmqldakapa
  61 hprplqllgr vgpgsrqlad gvnsgqglgi eiigtlqlvl cvlattdrrr rdlggsapla
121 iglsvalghl laidytgcgi nparsfgsav ithnfsnhwi fwvgpfigga lavliydfil
181 aprssdltdr vkvwtsgqve eydldaddin srvemkpk
SEQ ID NO: 4:
    1 mqsgmgwnvl dfwladgvns gqglgieiig tlqlvlcvla ttdrrrrdlg gsaplaigls
  61 valghllaid ytgcginpar sfgsavithn fsnhwifwvg pfiggalavl iydfilaprs
121 sdltdrvkvw tsgqveeydl daddinsrve mkpk
SEQ ID NO: 5
    1 maseikkklf wravvaefla mtlfvfisig salgfnyple rnqtlvqdnv kvslafglsi
  61 atlaqsvghi sgahlnpavt lglllscqis ilravmyiia qcvgaivata ilsgitsslv
121 dnslgrndla hgvnsgqglg ieiigtlqlv lcvlattdrr rrdlggsapl aiglsvalgh
181 llaidytgcs inparsfgsa vltrnfsnhw ifwvgpfigg alavliydfi laprssdftd
241 rmkvwtsgqv eeydldaddi nsrvemkpk
```

1. A method of preventing or reducing radiation-induced salivary dysfunction in a subject, comprising:
(a) administering a vector encoding an aquaporin (AQP) protein to the subject, and
(b) administering ionizing radiation to the subject following (a),
thereby preventing or reducing radiation-induced salivary dysfunction in the subject.

2. (canceled)

3. The method of claim 1, wherein the vector is administered to a salivary gland of the subject.

4. The method of claim 1, wherein AQP protein comprises an AQPI protein.

5. The method of claim 4, wherein the AQP1 protein comprises human AQP1 protein.

6. The method of claim 1, wherein the vector comprises a viral vector.

7. The method of claim 6, wherein the viral vector comprises an adenoviral vector.

8. The method of claim 7, wherein the adenoviral vector comprises serotype 2 or serotype 5.

9. The method of claim 6, wherein the viral vector comprises an adeno-associated viral (AAV) vector.

10. The method of 9, wherein the AAV vector comprises AAV2, AAV5, AAV6, AAV44.9, or BAAV.

11. The method of claim 9, wherein the AAV vector is administered as a virion comprising the AAV vector.

12. The method of claim 11, wherein the virion comprises an AAV virion.

13. The method of claim 1, wherein salivary gland function is maintained at a level equivalent or at least equivalent to salivary gland function prior to administration of the ionizing radiation.

14. The method of claim 1, wherein the subject is a human patient.

15. The method of claim 14, wherein the patient has head and neck cancer.

16. The method of claim 10, wherein the AAV vector is administered as a virion comprising the AAV vector.

17. The method of claim 16, wherein the virion comprises an AAV virion.

18. The method of claim 3, wherein AQP protein comprises an AQPI protein.

19. The method claim 18, wherein the AQP1 protein comprises human AQP1 protein.


SEQUENCE LISTING (5 sequences)

SEQ ID NO: 1 - protein, 269 aa, Homo sapiens
MASEFKKKLFWRAVVAEFLATTLFVFISIGSALGFKYPVGNNQTAVQDNVKVSLAFGLSIATLAQSVGHISGAHLNPAVTLGLLLSCQISIFRALMYIIAQCVGAIVATAILSGITSSLTGNSLGRNDLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 2 - protein, 186 aa, Homo sapiens
MPGARPLPLVLVPQNTLAWMQLDAKAPAHPRPLQLLGRVGPGSRQLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 3 - protein, 218 aa, Homo sapiens
MFWTFGYEAVSPAGPSHLFASLLLGVLLTITFMPGARPLPLVLVPQNTLAWMQLDAKAPAHPRPLQLLGRVGPGSRQLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 4 - protein, 154 aa, Homo sapiens
MQSGMGWNVLDFWLADGVNSGQGLGIEIIGTLQLVLCVLATTDRRRRDLGGSAPLAIGLSVALGHLLAIDYTGCGINPARSFGSAVITHNFSNHWIFWVGPFIGGALAVLIYDFILAPRSSDLTDRVKVWTSGQVEEYDLDADDINSRVEMKPK

SEQ ID NO: 5 - protein, 269 aa, Mus musculus
MASEIKKKLFWRAVVAEFLAMTLFVFISIGSALGFNYPLERNQTLVQDNVKVSLAFGLSIATLAQSVGHISGAHLNPAVTLGLLLSCQISILRAVMYIIAQCVGAIVATAILSGITSSLVDNSLGRNDLAHGVNSGQGLGIEIIGTLQLVLCVLATTDRRRRDLGGSAPLAIGLSVALGHLLAIDYTGCSINPARSFGSAVLTRNFSNHWIFWVGPFIGGALAVLIYDFILAPRSSDFTDRMKVWTSGQVEEYDLDADDINSRVEMKPK